(19) United States
(12) Patent Application Publication
(10) Pub. No.: US 2018/0182471 A1
(43) Pub. Date: Jun. 28, 2018

(54) SYSTEM FOR TRANSFORMING PATIENT MEDICAL RECORD DATA INTO A VISUAL AND GRAPHICAL INDICATION OF PATIENT SAFETY RISK

(71) Applicant: Children's Hospital Medical Center, Cincinnati, OH (US)

(72) Inventors: Paul A. Yelton, California, KY (US); Frederick C. Ryckman, West Chester, OH (US); William Raymond Stone, Independence, KY (US); Stephen E. Muething, Batesville, IN (US); Eric Kirkendall, Batavia, OH (US)

(73) Assignee: Children's Hospital Medical Center, Cincinnati, OH (US)

(21) Appl. No.: 15/113,737

(22) PCT Filed: Jan. 23, 2015
(86) PCT No.: PCT/US2015/012635

§ 371 Date: Jul. 22, 2016

Related U.S. Application Data
(60) Provisional application No. 61/931,471, filed on Jan. 24, 2014.
(60) Provisional application No. 61/949,704, filed on Mar. 7, 2014.

(51) Int. Cl. G16H 10/60; G16H 50/30; G16H 50/70; G06F 17/30

(52) U.S. Cl. CPC G16H 10/60; G06F 17/30598; G16H 50/70; G16H 50/30

(57) ABSTRACT

Methods, apparatus, devices, and/or systems related to the transformation of patient medical record data into a visual and graphical indication of patient safety risk are provided. In some embodiments, the disclosed methods, systems, and/or apparatus relate to retrieving electronic health record(s) comprising patient data of one or more patients, classifying the one or more patients into one or more risk categories, and displaying a risk report on a communications device. Alternative embodiments may relate to receiving new patient data for a patient and updating the corresponding electronic health record in the patient database with the received new patient data. In some embodiments, when the electronic health record is updated with new patient data, the updating and retrieving steps may occur in real time, such that the updated electronic health record is immediately classified and reflected in a displayed risk report.

### CROSS-REFERENCE TO RELATED APPLICATIONS

The present application claims priority to U.S. provisional application Ser. No. 61/931,471, with a filing date of Jan. 24, 2014, and to U.S. provisional application Ser. No. 61/949,704, with a filing date of Mar. 7, 2014. The above-mentioned provisional applications are hereby incorporated by reference.

### TECHNICAL FIELD

The system of the present disclosure relates to the medical, medical monitoring, medical analysis, medical reporting, and medical center administration fields. This disclosure relates to the care and management of patients, including high-risk patients.

### BACKGROUND

The complex operations of a medical center, such as a hospital and units thereof, require real-time awareness of hundreds of data points. Leaders, from hospital administrators to charge nurses, need information in order to identify and mitigate risks related to: the flow of patients into, from, and within a medical facility; patient and family experiences, including past experiences and experiences with multiple services or units; safety, including safety risks to the patient and safety risks to the staff; and unit-level risks, including staffing levels and types of patients within units.

Managing a medical center is inherently risky. Additionally, risks in a medical center constantly change with patient flow and staffing levels, for example. Reducing and more effectively managing risk in a medical center can improve outcomes for patients; improve the experiences of patients, family, and medical center staff; and save time and money. There is a need to ensure that front-line medical center workers are more keenly aware of risks that may affect their job performances. Similarly, there is a need to ensure that medical center administrators are aware of the relative riskiness of medical center units in order to adequately manage risks as they appear. Furthermore, risks need to be quickly and accurately identified, and risk management steps need to be performed efficiently.

### SUMMARY

The present disclosure pertains to systems for identifying risks and, more particularly, to systems for transforming patient medical record data into a visual and graphical indication of patient safety risk. While the systems according to the present disclosure are configured to be used in any clinical setting having a plurality of patients and/or a plurality of workers, the embodiments of the current disclosure will be described for use in a hospital for exemplary purposes. The systems may be scaled down to smaller settings, such as a doctor's office or a rehabilitation clinic, or scaled up to, for example, a multi-facility health consortium.

Some example embodiments according to at least some aspects of the present disclosure may comprise methods, apparatus, devices, and/or systems pertaining to the transformation of patient medical record data into a visual and graphical indication of patient safety risk. Such a system may include a communications device, a patient database including electronic health record(s) comprising patient data of patient(s), and a computer processor instructed to: retrieve the electronic health record(s) of the patient(s), classify the patients according to risk, and display a risk report on the communications device. In some more detailed embodiments, the computer processor may be further instructed to receive patient data for a patient and update the electronic health record corresponding to that patient with the received patient data. In some embodiments, when a patient's electronic health record is updated, the computer processor may be configured to retrieve the updated electronic health record in real time, such that the risk report reflects patient data added to the electronic health record substantially in real time.

For the purpose of this disclosure, “real-time” refers to processing that produces results to a user that is perceived to be substantially immediate, or at least within ten or fewer seconds. “Near real-time” produces results to a user within a reasonable expected wait time, which is typically less than a minute. Additionally, for the purpose of the present disclosure, “unit” refers to a subdivision of a medical center. For example, a unit may be a geographic subdivision, such as a floor, wing, etc. “Unit” may also refer to a particular medical specialty, wherein patients classified in a particular unit may receive similar care and/or have similar medical conditions.

Some example embodiments may be configured to identify and display risks associated with a particular patient to ensure that front-line staff are aware of such patient risks during or immediately following a shift change, for example. Other example embodiments may be configured to identify and display risks associated with a particular unit, which may, for example, assist a unit's internal management in ensuring that the appropriate staff are available to care for the patients in the unit. Additionally, example embodiments may be configured to identify and display risks associated with a medical center at-large, which may allow an administrator, for example, to shift resources from a relatively low-risk unit to a relatively high-risk unit in order to optimally manage present or expected risks within the medical center.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings.

FIG. 1 shows an illustration of an example network environment for a system for transforming patient medical record data into a visual and graphical indication of patient safety risks;

FIG. 2 shows an illustration of another example network environment for a system for transforming patient medical record data into a visual and graphical indication of patient safety risks;

FIG. 3 is a flowchart showing an exemplary method for transforming patient medical record data into a visual and graphical indication of patient safety risks;

FIG. 4 is an exemplary risk report at the institutional level;

FIG. 5 is an exemplary risk report at the unit level; and

FIG. 6 is an exemplary risk report at the patient level.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description and drawings are not meant to be limiting. Other embodiments may be utilized, and other changes may be made, without departing from the spirit and scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and constitute part of this disclosure.

The present disclosure is drawn to methods, systems, devices, and/or apparatus related to the transformation of patient medical record data into a visual and graphical indication of patient safety risk. More specifically, the disclosed methods, systems, and/or apparatus relate to retrieving electronic health record(s) comprising patient data of patient(s), classifying the patient(s) into one or more risk categories, and displaying a risk report on a communications device. Alternative embodiments may further relate to receiving new patient data for a patient and updating the corresponding electronic health record in the patient database with the received new patient data. In some embodiments, when the electronic health record is updated with new patient data, the updating and retrieving steps may occur in real time, such that the updated electronic health record is immediately classified and reflected in a displayed risk report.

The present disclosure contemplates that, in certain embodiments, medical centers may already implement electronic health records or have plans to do so. Electronic health records may simplify record keeping in a medical center by storing all pertinent patient information in a database that may be called upon by a range of medical center staff for a variety of purposes, not the least of which includes the day-to-day care and treatment of a patient. Exemplary information that may be contained in an electronic health record includes the following: administrative and billing data, patient demographics, progress notes, vital signs, medical histories, diagnoses, medications, immunizations, allergy information, radiology images, and lab and test results. Those skilled in the art will realize that the above description is merely a non-limiting example and that, generally, an electronic health record may contain comprehensive health-related information about a patient and the provision of health services thereto. Electronic health records are useful within a medical center to provide up-to-date patient information to a variety of medical center workers, and they may be further useful to provide up-to-date patient information to various healthcare providers across multiple medical centers.

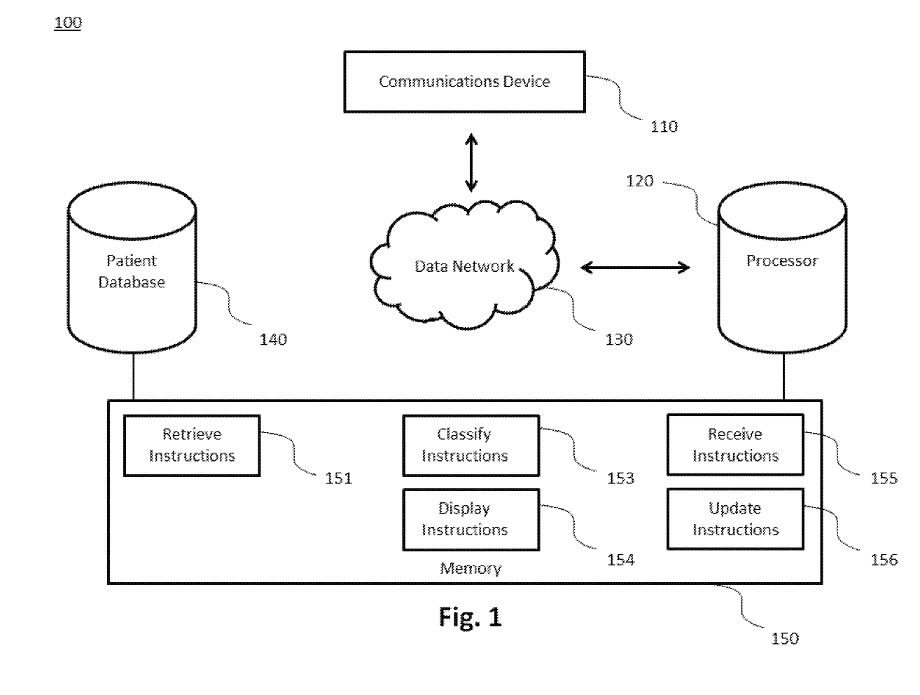

FIG. 1 shows an example risk identification and display network environment 100 according to at least some aspects of the present disclosure. Risk identification and display network environment 100 includes a data network 130, a communications device 110, a processor 120, a memory 150, and a patient database 140. Communications device 110 may connect to a computer system that includes processor 120, memory 150, and patient database 140 via data network 130 (data networks may be direct connections, indirect connections, network connections, interne connections, wired connections, wireless connections, cellular connections, and the like facilitating the exchange of data between the components).

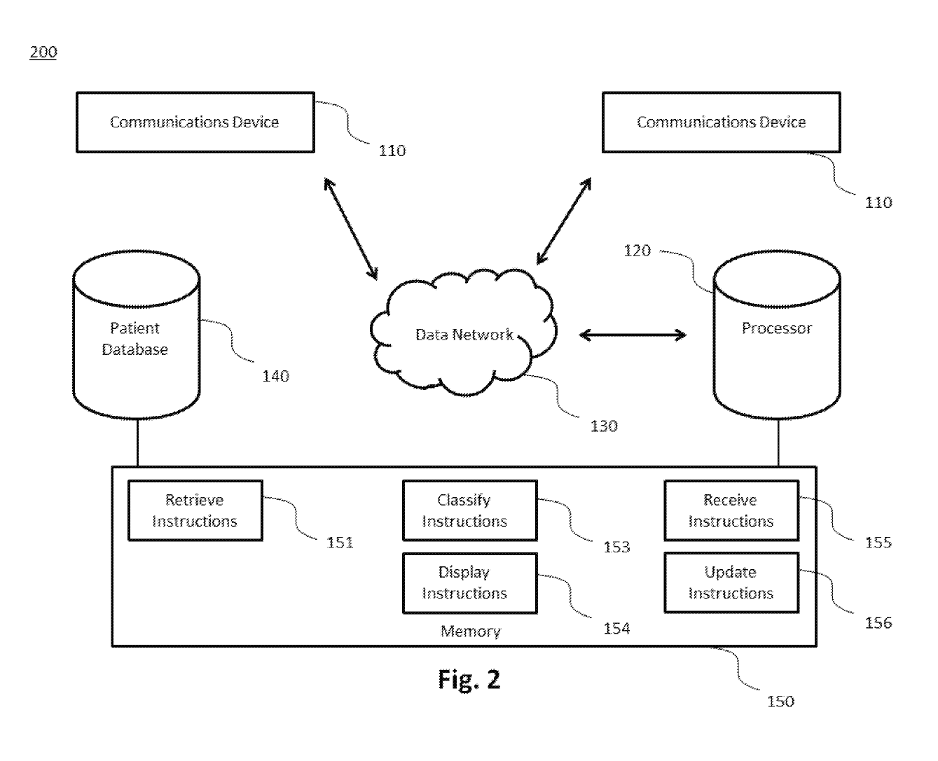

While FIG. 1 shows a single communications device 110 in risk identification and display network environment 100, multiple communications devices 110 may connect to the computer system. FIG. 2 shows an alternate risk identification and display network environment 200 according to at least some aspects of the present disclosure. Alternate risk identification and display network environment 200 includes a data network 130, multiple communications devices 110, a processor 120, a memory 150, and a patient database 140. Communications devices 110 may connect to a computer system that includes processor 120, memory 150, and patient database 140 via data network 130.

Communications device 110 generally has a display and a graphical user interface. More specifically, communications device 110 may represent one or more of a personal computer; a desktop computer; a laptop computer; a mobile telephone; a smart telephone; a portable computing device; a wearable computing device, such as a computing device incorporated within a pair of glasses, a watch, and the like; a tablet device; or any other suitable communications device that will be apparent to those skilled in the art. In an embodiment, communications device 110 may be a device such as a desktop computer, a laptop computer, or a tablet device common in or easily compatible in a medical center such as a hospital. The system of the present disclosure may be accessed on a stand-alone application, on the web accessed by a web browser, and the like.

In an exemplary embodiment, an electronic health record in patient database 140 may include the following patient data: administrative and billing data, patient demographic data, personal data, progress notes data, vital signs data, medical history data, diagnosis data, treatment plan data, medications data, immunization data, allergy data, data corresponding to radiology images, lab and test results data, and any other data that may combine to provide comprehensive, health-related information about a patient. In an additional detailed embodiment, for the purposes of the present disclosure, patient database 140 may include medical center data. Medical center data may correspond to the operations of a medical center or a unit thereof, such as staffing data, capacity data, or other data that may pertain to providing healthcare services to and/or treating one or more patients.

An electronic health record for a patient may be stored in the patient database 140, which may also contain electronic health records for a plurality of patients. Furthermore, electronic health records may be stored in a plurality of patient databases 140. In additional detailed embodiments where the system retrieves additional data, such as medical center data, the additional data may be likewise stored in patient database 140 or in a plurality of patient databases 140. However, for the sake of simplicity, electronic health records and any other data are represented in the figures as being contained within one patient database 140.

In an embodiment, as shown in FIG. 1 and FIG. 2, memory 150 may include retrieve instructions 151, classify instructions 153, display instructions 154, receive instructions 155, and update instructions 156. The instructions on memory 150, when executed by processor 120, may cause processor 120 to perform certain tasks, each of which will be described below.

The processor 120 in accordance with the present disclosure may execute retrieve instructions 151 contained on memory 150 to retrieve one or more electronic health records of one or more patients from patent database 140. In an embodiment, an electronic health record of only one patient may be retrieved. In an alternate embodiment, a plurality of electronic health records corresponding to more than one patient, but less than all patients in a medical center, may be retrieved. In still another embodiment, a plurality of electronic health records corresponding to all patients in a medical center may be retrieved. In some detailed embodiments, retrieve instructions 151 may additionally direct processor 120 to retrieve medical center data from patient database 140.

The processor 120 in accordance with the present disclosure may execute classify instructions 153 contained on memory 150 to classify one or more patients into one or more risk categories based on the retrieved electronic health records. Classify instructions 153 may utilize one or more algorithms and/or methods to analyze retrieved electronic health records and to classify the patients corresponding to the retrieved electronic health records into the one or more risk categories. In further embodiments, classify instructions 153 may utilize one or more algorithms and/or methods to analyze retrieved medical center data along with retrieved electronic health records, particularly for, but not limited to, classifying the riskiness of a unit. In a detailed embodiment, classify instructions 153 may utilize one or more algorithms programmed in JavaScript frameworks, including AngularJS, Knockout.js, Bootstrap, and/or Toastr.

In an embodiment, classify instructions 153 may cause the processor 120 to classify one or more patients into one or more risk categories. In a more detailed embodiment, the risk categories may include a situational awareness category, a patient-family experience category, a flow category, a safety category, and/or a unit health category. Each risk category comprises one or more data indicators. In an embodiment, each data indicator may correspond to data that may be found in an electronic health record. In another embodiment, some data indicators may correspond to data that may be found in an electronic health record, while other data indicators may correspond to data that may be found in medical center data. For example, one or more data indicators under the unit health risk category may correspond to data that may be found in medical center data. By associating data that may be found in an electronic health record and/or in medical center data with data indicators, one or more patients may be classified into the one or more risk categories. In the same way, a unit may be scored or otherwise classified according to risk. Those skilled in the art will realize that the following data indicators and their descriptions merely serve to illustrate the type of data indicators that each risk category comprises. Furthermore, different medical centers may utilize different data indicators, by necessity, by preference, or by the nature of the particular medical center. It is expressly contemplated by the present disclosure that each risk category shall encompass any such different data indicator which, by the nature of the risk category, patterns in the following groupings of data indicators, and/or the descriptions of each data indicator, would most naturally fall within that risk category. It is also expressly contemplated by the present disclosure that a particularly high-risk data indicator may be encompassed by, and/or cause a patient to be classified in, more than one risk category; for example, a single data indicator could conceivably cause a patient to be classified in both the situational awareness risk category and the safety category.

The situational awareness risk category may include a family concern for safety data indicator. Generally, a family concern for safety data indicator may correspond to a family or a member thereof being upset or unsatisfied with a patient's care or concerned with a perceived safety issue. The situational awareness risk category may also include a missed medication data indicator, a delayed medication data indicator, a missed feeding data indicator, a delayed feeding data indicator, a cancelled surgery data indicator, a delayed procedure data indicator, a delayed diagnosis data indicator, an intravenous (IV) issue data indicator, and/or a high-risk therapy indicator. The significance of the above-mentioned data indicators will be apparent to those skilled in the art. The situational awareness risk category may also include a readmission data indicator. A patient may be classified under a readmission data indicator if the patient was previously discharged and readmitted to the medical center or a unit thereof within a pre-defined period of time. Preferably, a patient may be classified under a readmission data indicator if the patient was previously discharged and readmitted to the medical center or a unit thereof within a week of discharge. The situational awareness risk category may further include a high early warning score data indicator. Those skilled in the art will appreciate that different medical centers may have slightly different, though generally similar, standardized systems for assessing and responding to deteriorating patients. The high early warning score data indicator is intended to encompass any high score, as defined by the particular medical center and/or geographic jurisdiction, that would alert clinicians to take action on a patient, including more frequent observations, admission to an intensive care unit, evaluation by a medical emergency team, or other like escalated level of care. Some examples may include a modified early warning score (MEWS), a pediatric early warning score (PEWS), a national early warning score (NEWS), and other similar systems. In a detailed embodiment, the system of the disclosure may automatically monitor patient data, such as vitals, and trigger a high early warning score data indicator, a subjective degrading patient data indicator, and/or a related data indicator; such a protocol may be defined by the particular medical center. The situational awareness category may also include a subjective degrading patient indicator. A patient may be classified under a subjective degrading patient indicator if a clinician interacting with the patient, e.g., a treating physician or a bedside nurse, indicates that he feels as if the patient's condition is worsening, regardless of any objective criteria. The subjective degrading patient indicator generally corresponds to a clinician's personal medical or clinical judgment.

The patient-family experience risk category may include a family concern for care data indicator. A family concern for care data indicator may correspond to a family or a member thereof being upset or unsatisfied with a patient's care. The patient-family experience risk category may include a surgical add-on data indicator. A patient may be classified under a surgical add-on data indicator if a patient is scheduled for one or more procedures that require the patient to not eat or drink for a certain amount of time. Delays in the procedures, thereby lengthening the time of the food or drink restriction, could cause issues for the patient and/or the family of the patient. The patient-family experience category may include a communication data indicator. A patient may be classified under a communication data indicator if the patient or family experienced communication-related problems in the past or if the potential for communications problems is high, such as in complex treatment plans or with patients being treated by a large number of clinicians. The patient-family experience risk category may include a multiple consults data indicator. A patient may be classified under a multiple consults data indicator if the patient has multiple treatment teams during the course of a single stay. The multiple consults data indicator may serve to alert all clinicians that others may be treating the patient so the teams can more effectively coordinate and avoid taking actions that may conflict with the treatment of another team. The patient-family experience risk category may additionally include an off normal unit data indicator. A patient may be classified under an off normal unit data indicator if the patient is placed on a unit that may not normally care for the patient specifically or the type of patient generally. Such a situation can occur if a patient develops a particular condition requiring temporary treatment in another unit, if the normal unit is overcrowded or understaffed, or in other like scenarios that will be apparent to those skilled in the art. The patient-family experience risk category may include a high pain score data indicator. A patient may be classified under a high pain score data indicator if the patient self-reports high pain sensations, a clinician observes or scores high pain sensations, a physiological assessment (e.g., a brain scan) indicates high pain sensations, combinations thereof, or any manner accepted by a medical center to indicate pain and/or take pain into account when making medical or clinical judgments. The patient-family experience risk category may further include a not-by-mouth data indicator. A patient may be classified under a not-by-mouth data indicator if the patient is under a not by mouth, nil by mouth, nil per os, or other like medical order that prohibits anyone from providing food, drink, or medications by mouth. Such an order may occur before the administration of general anesthesia, for certain medical conditions, and after some types of surgery. The patient-family experience risk category may include a length of stay data indicator, under which a patient may be classified if the patient is expected to stay in the medical center or a particular unit thereof for more than a predetermined length of time, such as one week or one month, as determined by the medical center. In an alternate embodiment, all inpatients may be classified under a length of stay data indicator, the data indicator further specifying the expected length of stay. The patient-family experience risk category may include a previous experience failure data indicator, under which a patient may be classified if the patient previously stayed at the medical center and experienced an issue that may be relevant to the current stay. Additionally, the patient may be classified under a transferred from different unit data indicator and/or a transferred from critical unit data indicator; the significance of these data indicators will be readily apparent to those skilled in the art.

The flow risk category may include a pending admission data indicator. A patient may be classified under a pending admission data indicator if it is expected that the patient will soon be admitted to the medical center and/or soon be admitted to a particular unit. The pending admission data indicator may indicate when the patient is expected to be admitted and/or to what unit the patient is expected to be admitted. In a detailed embodiment, a patient may be classified under the pending admission data indicator if a patient is being admitted to the hospital initially or for a scheduled admission, is being admitted from the emergency department, is admitted from a post-anesthesia care unit after a surgery, and the like. The flow risk category may include a predicted discharge data indicator. A patient may be classified under a predicted discharge data indicator if a predicted discharge date is established and/or it is a predetermined length of time away from a previously-established predicted discharge data, such as one week, two days, one day, the next 12-18 hours, or the next four hours, for example. The pending admission data indicator and/or the predicted discharge data indicator may also be used to predict the medical center's capacity or the capacity of a unit thereof at some date in the future. The flow risk category may also include an overnight hold data indicator. A patient may be classified under an overnight hold data indicator may be held in a unit or department longer than initially planned, such as in the emergency department, in an intensive care unit, in a post-anesthesia care unit, and the like. Additionally, the flow risk category may include a unit capacity data indicator. Preferably, a unit capacity data indicator would be accessible from an institutional-level risk report and/or a unit-level risk report and may comprise current unit capacity, projected future unit capacity (including expected admissions and expected discharges), and the like. A unit capacity data indicator may correspond to data from a plurality of sources, for example, from a plurality of electronic health records. The flow risk category may include a medically ready discharges data indicator, under which a patient may be classified when a physician determines the patient is ready to be discharged. Such a data indicator may correlate with hospital-set or accrediting organization-set standards for discharging medically ready patients within a set period of time, for example, within two hours.

The safety risk category may include an aggressive patient data indicator. A patient may be classified under an aggressive patient data indicator if the patient has recorded instances of an aggressive and/or violent nature toward others in the past; such patients may require a specially-trained team to assist in providing care. The safety risk category may also include a self-harm data indicator, under which some patients may be classified if the patients might be at risk of harming themselves, for example, some psychiatric patients or patients who clinicians subjectively believe may be at such risk. The safety risk category may include a potential security risk data indicator. A patient may be classified under a potential security risk data indicator if the patient, for example and without limitation, was admitted from prison, has a known or suspected gang affiliation, has historically aggressive parents, has a dysfunctional family situation, and/or is aggressive. The safety risk category may include a line-drain-airway data indicator. A patient may be classified under a line-drain-airway data indicator if the patient has an active line, drain, tube, other item leading into or out of the inside of the patient's body. Generally, this may include patients with a surgical drain used to remove pus, blood, or other fluids from a wound or opening; a tracheotomy; an ostomy; a ventilator tube; a chest tube; a catheter; a nasogastric tube; a nasojejunal tube; a gastrojejunal tube; a jejunal tube; an endotracheal tube; intravenous lines; implantable ports; and the like. Such patients may be particularly susceptible to infection and/or may require additional or more attentive care. The safety risk category may include a signed and held order data indicator. A patient may be classified under a signed and held data indicator if, e.g., a physician places an order prior to the patient arriving on unit, such as while the patient is still in the emergency department awaiting transfer to another unit. The safety risk category may include a prevention standards data indicator. A patient may be classified under a prevention standards data indicator if the patient has a condition or is undergoing treatment for which the medical center has a defined protocol that clinicians must follow with respect to the patient. For example and without limitation, there may be a ventilator associated respiratory infection (VARI) prevention standard, a catheter associated urinary tract infection (CAUTI) prevention standard, a central line blood stream infection (CLBSI) prevention standard, and a pressure ulcer (PU) prevention standard. The prevention standards data indicator can alert clinicians to patients with an active prevention standard and can alert administrators to units having a large portion of prevention standards patients. The safety risk category may include a critical lines and tubes indicator. A patient may be classified under a critical lines and tubes data indicator if the patient has an active line or tube; more particularly, this data indicator may be used if a line or tube is not considered critical for most patients but is critical for a particular patient, and/or when a patient is undergoing multiple procedures from multiple teams. Additionally, certain medical orders, such as “give medications via nasojejunal tube,” may cause a patient to be classified under a critical lines and tubes indicator. Generally, this data indicator serves to alert clinicians that a particular line or tube is critical for survival and/or to prevent serious injury and should trigger that clinician to investigate further before taking action with regard to the line or tube at issue. The safety risk category may also include a multiple services data indicator. A patient may be classified under a multiple services data indicator if the patient is being treated by more than one team. The multiple services data indicator may serve to alert all clinicians that others may be treating the patient, so the teams can more effectively coordinate and avoid taking actions that may conflict with the treatment of another team. The safety risk category may also include a subjective degrading patient data indicator and a high early warning score data indicator; both data indicators are described in the discussion of the situational awareness risk category and apply similarly to the safety risk category. The safety risk category may also include a multiple medications data indicator and a reactive medications data indicator; those skilled in the art will rapidly recognize the significance of these data indicators. The safety risk category may include a seclusion order data indicator, a restraint order data indicator, a private room data order indicator, an imminent danger order data indicator, and/or a continuous observation order data indicator. Those skilled in the art will know the significance of these data indicators.

In additional detailed embodiments, the system of the present disclosure may include a unit health risk category. The unit health risk category may include a staffing level data indicator. The staffing level data indicator may provide leaders with real-time indications of who is scheduled to work, what types of people (e.g., physicians, nurses, techs, etc.) are scheduled to work at a given time, whether a scheduled worker has called in sick, and the like. The staffing level data indicator may enable leaders to move staff from over-staffed units to under-staffed units based on real-time data. Furthermore, the staffing level data indicator may trigger alerts, such as when the proportion of doctors or nurses to patients reaches a predefined point. The unit health risk category may include a unit capacity data indicator. The unit capacity data indicator may indicate on a unit-by-unit basis how many beds are occupied versus how many beds are open, either in real time or at a projected future time. The unit health risk category may also include a staff on call data indicator, which may, in conjunction with the staffing level data indicator, assist leaders in determining the level of back-up support a unit or an institution has in case of an emergency or unpredicted increase in patients and/or risk. The unit health risk category may include an isolation indicator. Patients requiring isolation may take up more unit space and/or require specialized staff to be present. Having real-time knowledge of isolation patients may reduce unit-level risk or institutional-level risk. For example and without limitation, a patient who may normally be transferred to one unit from the emergency department may be transferred to another unit if the first unit has a large number of isolation patients occupying the staff at a given time. The unit health risk category may also include an overall unit risk score data indicator, which may be based on a scoring algorithm with the goal of identifying the highest risk units containing the sickest and/or riskiest patients. In a detailed embodiment, the overall unit risk score data indicator may be used at an institutional level to sort and view the units from most risky to least risky.

The processor 120 in accordance with the present disclosure may execute display instructions 154 contained on memory 150 to display a risk report on communications device 110. Display instructions 154 may cause a risk report to be displayed on communications device 110 at one of a plurality of levels or views. In an embodiment, a risk report may be displayed at an institutional level, a unit level, or a patient level. A user of communications device 110 may switch between the various levels. For example, a medical center administrator may view an institutional level risk report, a unit level risk report, and/or a patient level risk report. In an alternate embodiment, a user of communications device 110 may be able to view a risk report at one or more, but not all, of the plurality of levels. For example, a charge nurse may be able to view a risk report at a unit level and a patient level, but not at an institutional level. Such permissions may be controlled by the medical center at its discretion. The above description of a risk report envisions the system of the present disclosure to be used in a medical center comprising a primary location made up of a plurality of units, each of which may have a plurality of patients. It is important to note that many medical centers have expanded to an enterprise system of a plurality of locations, each of which may function semi-autonomously day-to-day, either through mergers or organic expansion. The present disclosure expressly contemplates the present system functioning in such an expanded medical center. For example and without limitation, a risk report may comprise a selectable menu listing each location; such a menu may comprise key, up-to-date risk information for that location and the enterprise as a whole. In an alternate embodiment, such a functional menu may be referred to as an enterprise level of a risk report, through which an institutional level risk report for each location may be selected by communications device 110.

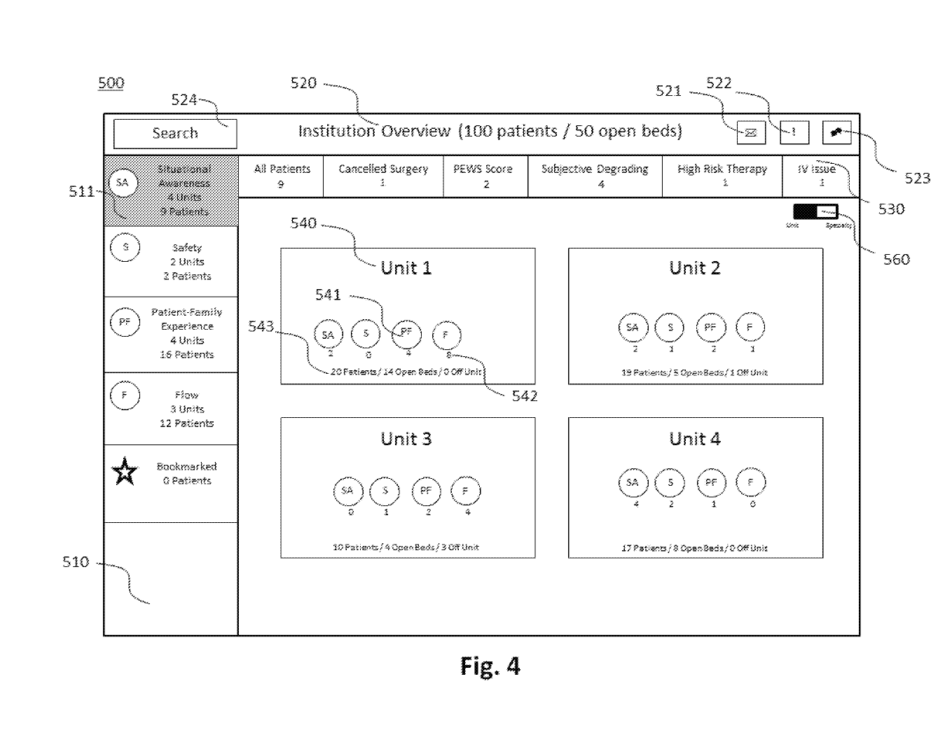

In an embodiment, display instructions 154 may cause the processor 120 to display a risk report on a communications device 110, said risk report being displayed at an institutional level. FIG. 4 shows an example risk report at an institutional level according to at least some aspects of the present disclosure. In an embodiment, institutional-level risk report 500 may include a menu of toggleable risk category icons 510, a menu of toggleable data indicator icons 530, and a plurality of unit representations 540, wherein each unit representation 540 corresponds to a unit in the medical center and includes a risk score 542 for each risk category 541. Each unit representation 540 may refer to a geographic subdivision of a medical center and/or a medical specialty. In an additional detailed embodiment, institutional-level risk report 500 may include a toggleable switch 560, which may determine whether each unit representation 540 refers to a geographic subdivision of a medical center or a medical specialty. Each risk score 542 for each risk category 541 corresponds to risk in the unit, which may be signified in each unit representation 540. In detailed embodiments, institutional-level risk report 500 may include a header 520, which may include information from one or more data indicators. For example, in FIG. 4, header 520 includes information representing the number of inpatients and the number of open beds in the institution at the moment (i.e., information corresponding to institutional capacity). Institutional-level risk report 500 may further include a search button 524, which may allow a user to search for a patient, such as by electronic health record number. In further detailed embodiments, each unit representation 540 may include unit information 543. For example and without limitation, unit information 543 may correspond to total patients on unit, total beds available on unit, and patients temporarily off unit. Menu of risk category icons 510 may contain a plurality of icons, each corresponding to a risk category. FIG. 4 further illustrates a toggled risk category icon 511 in menu of toggleable risk category icons 510. Toggling a risk category icon in menu of toggleable risk category icons 510 may cause menu of toggleable data indicator icons 530 to be displayed, such as underneath header 520 or underneath toggled risk category icon 511 as a sub-menu in menu of toggleable risk category icons 510. Each data indicator icon may indicate the number of patients classified under the particular data indicator. Toggling an icon in menu of data indicator icons 530 may filter each unit by data indicator. For example, the results of such a filter may be displayed by re-ordering each unit representation 540 by riskiness according to the toggled data indicator icon. In FIG. 4, no data indicator icon is toggled, so no filtering by data indicator has occurred. In a detailed embodiment, each data indicator icon may include real-time information corresponding to the number of patients classified under the data indicator. In an alternate detailed embodiment, a toggled risk category icon 511 may filter each unit representation 540 by risk category and/or cause menu of toggleable data indicator icons 530 to be displayed. For example and without limitation, toggling the flow risk category icon may sort each unit representation 540 according to patients flowing into or out of the unit and/or cause each unit representation 540 to display only information pertinent to the flow risk category and data indicators therein. Filtering according to the flow risk category may cause each unit representation 540 to display a projected open beds indicator. In FIG. 4, information contained within each unit representation 540 is exemplary and may be displayed differently, such as through color-coding, in a different order, etc.

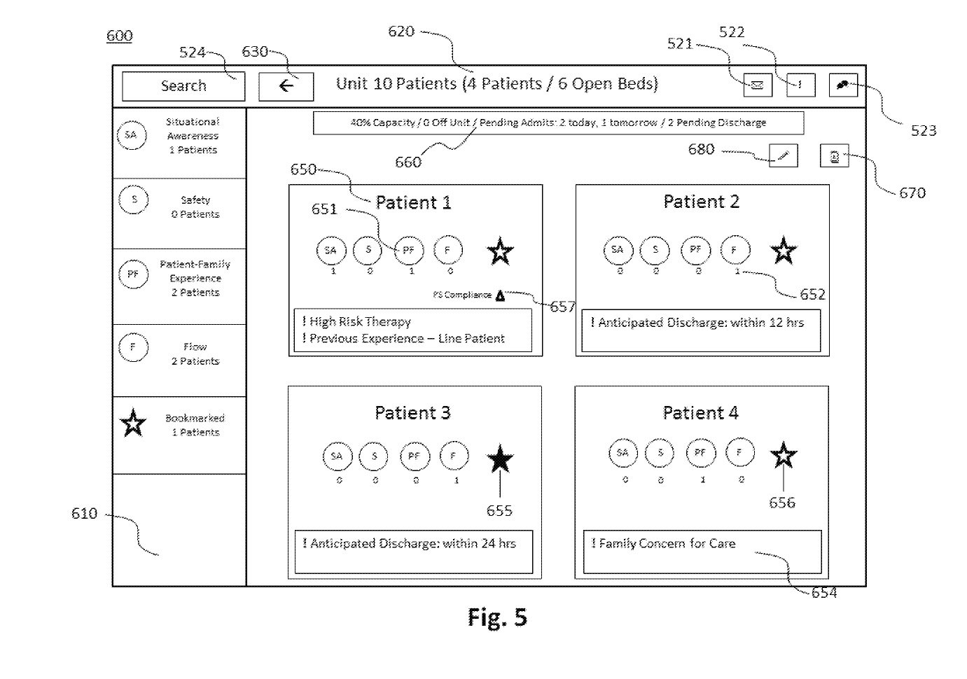

In an embodiment, display instructions 154 may cause the processor 120 to display a risk report on a communications device 110, said risk report being displayed a unit level. Unit may refer to a particular geographic subdivision of a medical center, or it may correspond to a particular medical specialty of the medical center. FIG. 5 shows an example risk report at a unit level according to at least some aspects of the present disclosure. In an embodiment, unit-level risk report 600 may include a menu of toggleable risk category icons 610 and a plurality of patient representations 650, wherein each patient representation 650 corresponds to a patient on the unit and may include a risk score 652 for each risk category 651 and a data indicator display 654. Each risk score 652 for each risk category 651 corresponds to risk in the patient, which is signified in each patient representation 650. Data indicator display 654 may display one or more data indicators under which the patient is classified (or risk-related information corresponding thereto). In an embodiment, each patient representation may include information indicating bed or room number, medical record number, the patient's name, the medical team (or specialty) to which the patient is assigned, and/or other associated information. In a detailed embodiment, unit-level risk report 600 may include a header 620, which may indicate the unit and/or include information from one or more data indicators. For example, in FIG. 5, header 620 includes information representing the number of inpatients and the number of open beds on the unit at the moment (i.e., information corresponding to unit capacity). In an alternate detailed embodiment, unit-level risk report 600 may include a sub-header 660, which may include, for example and without limitation, information corresponding to patients temporarily off unit, expected admissions to the unit, expected discharges from the unit, and the like. Unit-level risk report 600 may further include a search button 524, which may allow a user to search for a patient, such as by electronic health record number. FIG. 5 illustrates a menu of toggleable risk category icons 610 without any icon toggled. As with the institutional-level risk report, a toggled risk category icon may filter each patient by risk category and/or cause a menu of toggleable data indicator icons to be displayed (which may enable a user to filter each patient by data indicator). FIG. 5 is presented without a toggled risk category icon and without a menu of toggleable data indicator icons for contrast with FIG. 4; a toggled risk indicator icon, as in FIG. 4, may cause a similar menu of data indicator icons to be similarly displayed. For example and without limitation, toggling the flow risk category icon may sort each patient representation 640 according to each patient's flow activity and/or cause each patient representation 640 to display only information pertinent to the flow risk category and data indicators therein. In an additional detailed embodiment, a user may edit the data indicators of the flow risk category under which the patient is classified, for example, to indicate a predicted transfer or discharge. In an alternate detailed embodiment, patient representations 650 may include a prevention standards compliance icon 657. A patient representation 650 would display prevention standards compliance icon 657 only if the patient was classified under a prevention standards data indicator (in the safety risk category). Prevention standards compliance icon 657 may indicate whether or not the patient is compliant with one or more prevention standards, such as through a color change or other graphical depiction. Unit-level risk report 600 may include a back button 630, the selection of which may cause institutional-level risk report 500 to be displayed. Unit-level risk report 600 may also include a report button 670, the selection of which may cause summarized information about the unit to be displayed, including information useful for reporting at daily unit-level meetings amongst unit staff. In a detailed embodiment, unit-level risk report 600 may include a note button 680, the selection of which may cause a note page to be displayed where a user may add brief notes. In a detailed embodiment, notes are not a part of any patient's electronic health record and may automatically delete after a certain amount of time, such as a week.

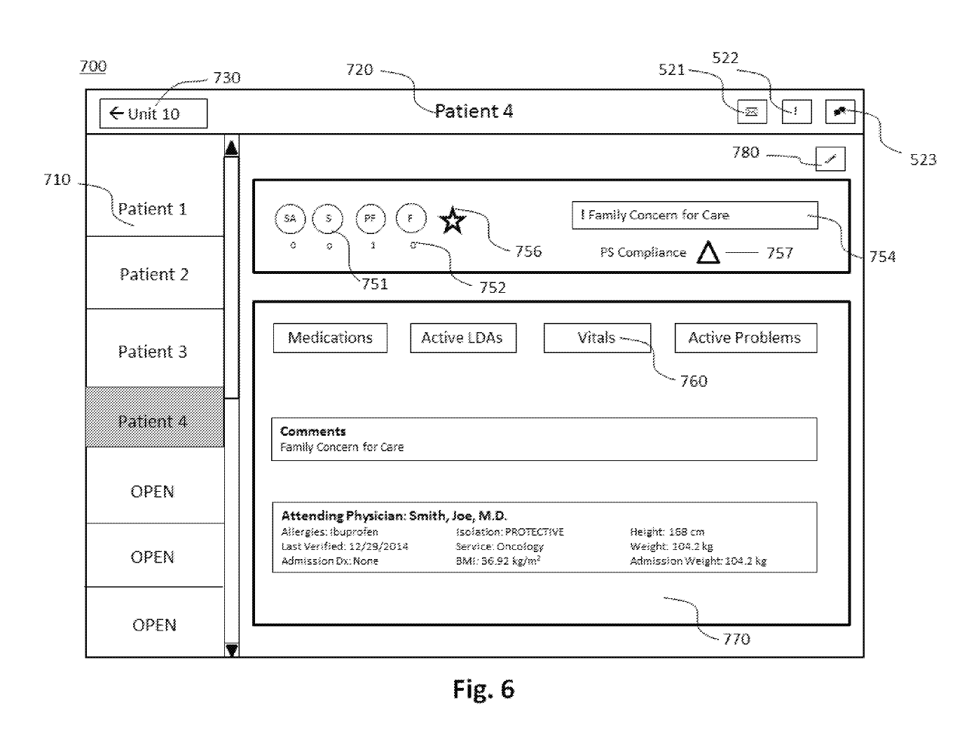

In an embodiment, display instructions 154 may cause the processor 120 to display a risk report on communications device 110, said risk report being displayed at a patient level. Information on a patient-level risk report may be displayed on one page, or it may be displayed on more than one page. FIG. 6 shows an example risk report at a patient level according to at least some aspects of the present disclosure. In an embodiment, patient-level risk report 700 may include a selectable list 710 of other patients on the unit, a risk score 752 for each risk category 751, and the electronic health record 770 of the patient and/or an electronic health record portion 760. Any information in a patient's electronic health record may be displayed in electronic health record portion 760. In a detailed embodiment, information in a patient's electronic health record 770 may be edited by a user. Risk score 752 for each risk category 751 corresponds to the risk of the patient. Data indicator display 754 may display one or more data indicators under which the patient is classified (or risk-related information corresponding thereto). In a detailed embodiment, the patient-level risk report 700 may include a header 720, which may indicate the patient, and/or a selectable unit icon 730, which may display the unit-level risk report for the unit in which the patient is located. Exemplary information that may be included in patient-level risk report 700 includes, but is not limited to, risk category and data indicator information, patient-level notes, assessment information, plan and body systems information, lines/drains/airways information, problems information, active medications information, vitals recordings and/or trends, treating clinicians, treating medical specialty, patient height and/or weight, patient allergies, etc. In a detailed embodiment, patient-level risk report 700 may include a selectable prevention standards compliance icon 757 if the patient is classified under a prevention standards data indicator. Selectable prevention standards data indicator 757 may indicate whether the patient is compliant with one or more prevention standards. Selecting selectable prevention standards data indicator 757 may open a window (or otherwise cause to be displayed), providing details on any applicable prevention standards for the patient and compliance thereto. In an embodiment, patient-level risk report 700 may include a note button 780, the selection of which may cause a note page to be displayed where a user may add notes. The medical center may choose whether or not notes entered on a note page accessed from a patient-level risk report 700 cause the patient's electronic health record in patient database 140 to be updated.

In an embodiment, header 520, 620, 720 may include a system messages button 521, a notifications button 522, and/or a user feedback button 523, as illustrated in FIG. 4, FIG. 5, and FIG. 6. Selection of system messages button 521 may cause information related to system downtimes, upgrades, and/or new functionality to be displayed. Selection of notifications button 522 may cause notifications triggered by certain data indicators to be displayed. For example, a patient classified under a subjective degrading patient data indicator may have a specialized treatment or observation plan that may expire after a set amount of time; the expiration of such a plan may cause a notification to be triggered, the notification being viewable when notifications button 522 is selected. Selection of user feedback button 523 may allow a user to give system feedback or functionality suggestions to the system administrator(s).

In an additional embodiment, a risk report may comprise a bookmark icon associated with each patient. A bookmark icon may be toggleable between an active state and an inactive state. A user may toggle a bookmark icon in the active state for a patient if the user thinks the patient requires special attention. In a detailed embodiment, a bookmark icon may be a way in which a clinician indicates a subjective degrading patient; in such an embodiment, toggling a bookmark icon to the active state may trigger a subjective degrading patient data indicator. In an alternate embodiment, a user may be required to log on to the system of the present disclosure with a unique username and password, and toggling a bookmark icon may add a patient to a bookmarked list viewable by only the particular user. A bookmark icon may take any desired shape; in FIGS. 4-6, the bookmark icon takes a star shape. A bookmark icon may be toggled between the active state and inactive state from one or both of the patient-level risk report 700 and unit-level risk report 600. In patient-level risk report 700, bookmark icon 756 may be located at one or more of on the selectable list of other patients and proximate to risk categories 751; bookmark icon 756 is represented in the inactive state. In unit-level risk report 600, bookmark icon 655, 656 may be located in patient representation 650 proximate to risk categories 651. FIG. 5 shows an active state bookmark icon 655 and an inactive state bookmark icon 656. As shown in FIG. 4 and FIG. 5, institutional-level risk report 500 and unit-level risk report 600, menu of toggleable risk category icons 510, 610 may further include a bookmark icon, which may function as a filter. In institutional-level risk report 500, toggling the bookmark icon from menu of toggleable risk category icons 510 may filter the displayed units by displaying only units having patients with a bookmark icon toggled in the active state. In unit-level risk report 600, toggling the bookmark icon from the menu of toggleable risk category icons 610 may filter the displayed patients by displaying only patients having a bookmark icon toggled in the active state.

The processor 120 in accordance with at least some aspects of the present disclosure may remain in real-time communication with patient database 140. By remaining in real-time communication with patient database 140, processor 120 may cause updates made to the electronic health records of one or more patients to be reflected in the risk report. Reflecting changes made to electronic health record(s) in the risk report may be important, since even small risk changes can have large impacts on how a medical center is managed. This importance magnifies as the size of the medical center (and the number of updates to electronic health records are made) increases. Details of how real-time connections are made and maintained are known to those skilled in the art.

In a detailed embodiment, patient-level risk report 700 may comprise a mechanism for receiving new patient data. For example, new patient data may be received via communications device 110, on which patient-level risk report 700 is displayed. Any action that triggers an input may cause the processor 120 to execute receive instructions 155 contained on memory 150 to receive new patient data by a graphical user interface of communications device 110. A clinician using the system of the present disclosure and viewing the risk report at the patient level on communications device 110 may enter information into electronic health record 770 or electronic health record portion 760 directly from communications device 110. For example, if communications device 110 is a tablet, tapping on electronic health record 770 or an electronic health record portion 760 may trigger an input mechanism, such as making text editable, providing a line for entering additional information in a particular category, and/or displaying an on-screen keyboard. In another non-limiting example, if communications device 110 is a desktop computer or a laptop computer, one or more electronic health record portions 760 may be clickable, whereupon a click triggers an input mechanism. Entering new patient data into an electronic health record is known to those skilled in the art.

In a detailed embodiment, the receipt of new patient data may cause processor 120 to execute update instructions 156 contained on memory 150. Update instructions 156, may cause the processor 120 to update the electronic health record of the patient to be updated with the received new patient data by updating patient database 140. In such an embodiment, the received new patient data would be displayed when the electronic health record of the patient is accessed, both within the system of the present disclosure and in any other standard system for accessing the electronic health record. In an additional embodiment, updating the electronic health record of the patient with received new patient data may cause the processor 120 to re-execute retrieve instructions 151, classify instructions 153, and display instructions 154 in real time. In this manner, received new patient data may be immediately reflected in the risk report.

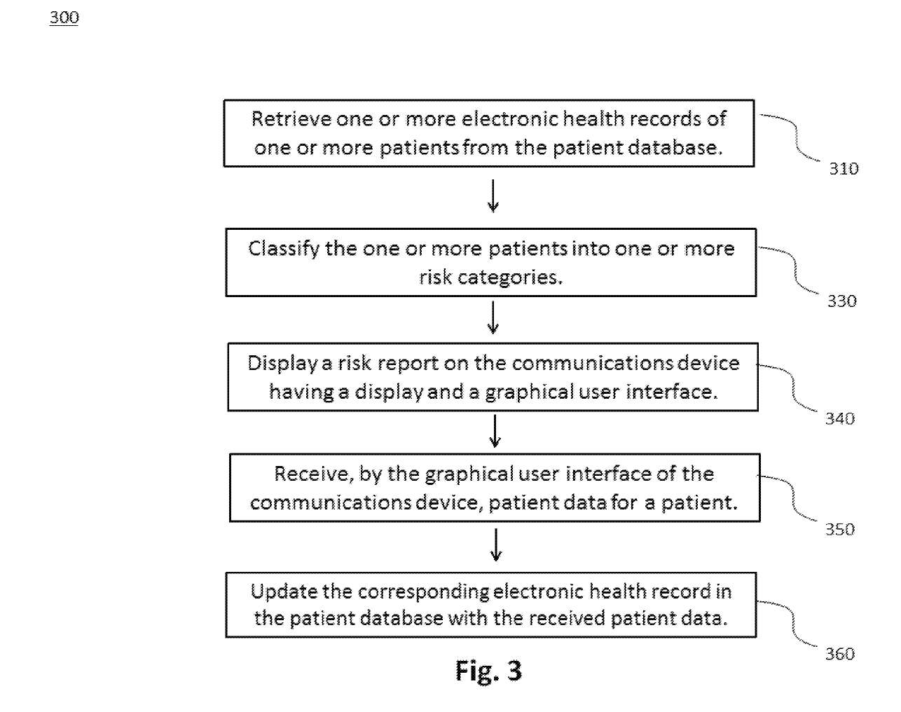

FIG. 3 is a flowchart showing an exemplary method 300 for transforming patient medical record data into a visual and graphical indication of patient safety risks. As shown in FIG. 3, method 300 begins at stage 310 when a processor retrieves one or more electronic health records of one or more patients from the patient database. For example, as shown in FIG. 1, processor 120 executes retrieve instructions 151 on memory 150 to retrieve one or more electronic health records of one or more patients from the patient database 140. Stage 310 may be performed by, for example, processor 120 executing retrieve instructions 151.

At stage 330, the processor classifies the one or more patients into one or more risk categories. As shown in FIG. 1, processor 120 executes classify instructions 153 to classify the one or more patients into one or more risk categories. Stage 330 can be performed by, for example, processor 120 executing classify instructions 153.

At stage 340, the processor displays a risk report on the communications device having a display and a graphical user interface. Referring to FIG. 1, stage 340 may be performed by, for example, processor 120 executing display instructions 154. Processor 120 executes display instructions 154 to display a risk report on communications device 110 having a display and a graphical user interface.

At stage 350, the processor receives, by the graphical user interface of the communications device, patient data for a patient. As shown in FIG. 1, stage 350 may be performed by, for example, processor 120 executing receive instructions 155. Processor 120 executes receive instructions 155 to receive, by the graphical user interface of communications device 110 (which communicates with processor 120 via data network 130), patient data for a patient.

At stage 360, the processor updates the corresponding electronic health record in the patient database with the received patient data. As shown in FIG. 1, stage 360 may be performed by, for example, processor 120 executing update instructions 156. Processor 120 executes update instructions 156 to update the corresponding electronic health record in patient database 140 with the received patient data.

To provide additional context for various aspects of the current disclosure, the following discussion is intended to provide a brief, general description of a suitable computing environment in which the various aspects of the current disclosure may be implemented. While example embodiments of the current disclosure relate to the general context of computer-executable instructions that may run on one or more computers, those skilled in the art will recognize that the embodiments also may be implemented in combination with other program modules and/or as a combination of hardware and software.

Generally, program modules include routines, programs, components, data structures, etc., that perform particular tasks or implement particular abstract data types. Moreover, those skilled in the art will appreciate that aspects of the inventive methods may be practiced with other computer system configurations, including single-processor or multiprocessor computer systems, minicomputers, mainframe computers, as well as personal computers, hand-held wireless computing devices, microprocessor-based or programmable consumer electronics, and the like, each of which can be operatively coupled to one or more associated devices. Aspects of the current disclosure may also be practiced in distributed computing environments where certain tasks are performed by remote processing devices that are linked through a communications network. In a distributed computing environment, program modules may be located in both local and remote memory storage devices.

A computer may include a variety of computer readable media. Computer readable media may be any available media that can be accessed by the computer and includes both volatile and nonvolatile media, removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media (i.e., non-transitory computer readable media) includes, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which may be used to store the desired information and which may be accessed by the computer.

An exemplary environment for implementing various aspects of the current disclosure may include a computer that includes a processing unit, a system memory and a system bus. The system bus couples system components including, but not limited to, the system memory to the processing unit. The processing unit may be any of various commercially available processors. Dual microprocessors and other multi-processor architectures may also be employed as the processing unit.

The system bus may be any of several types of bus structure that may further interconnect to a memory bus (with or without a memory controller), a peripheral bus, and a local bus using any of a variety of commercially available bus architectures. The system memory may include read only memory (ROM) and/or random access memory (RAM). A basic input/output system (BIOS) is stored in a non-volatile memory such as ROM, EPROM, EEPROM, which BIOS contains the basic routines that help to transfer information between elements within the computer, such as during start-up. The RAM may also include a high-speed RAM such as static RAM for caching data.

The computer may further include an internal hard disk drive (HDD) (e.g., EIDE, SATA), which internal hard disk drive may also be configured for external use in a suitable chassis, a magnetic floppy disk drive (FDD), (e.g., to read from or write to a removable diskette) and an optical disk drive, (e.g., reading a CD-ROM disk or, to read from or write to other high capacity optical media such as the DVD). The hard disk drive, magnetic disk drive and optical disk drive may be connected to the system bus by a hard disk drive interface, a magnetic disk drive interface and an optical drive interface, respectively. The interface for external drive implementations includes at least one or both of Universal Serial Bus (USB) and IEEE 1394 interface technologies.

The drives and their associated computer-readable media may provide nonvolatile storage of data, data structures, computer-executable instructions, and so forth. For the computer, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the current disclosure.

A number of program modules may be stored in the drives and RAM, including an operating system, one or more application programs, other program modules and program data. All or portions of the operating system, applications, modules, and/or data may also be cached in the RAM. It is appreciated that the invention may be implemented with various commercially available operating systems or combinations of operating systems.

It is within the scope of the disclosure that a user may enter commands and information into the computer through one or more wired/wireless input devices, for example, a touch screen display, a keyboard and/or a pointing device, such as a mouse. Other input devices may include a microphone (functioning in association with appropriate language processing/recognition software as known to those of ordinary skill in the technology), an IR remote control, a joystick, a game pad, a stylus pen, or the like. These and other input devices are often connected to the processing unit through an input device interface that is coupled to the system bus, but may be connected by other interfaces, such as a parallel port, an IEEE 1394 serial port, a game port, a USB port, an IR interface, etc.

A display monitor or other type of display device may also be connected to the system bus via an interface, such as a video adapter. In addition to the monitor, a computer may include other peripheral output devices, such as speakers, printers, etc.

The computer may operate in a networked environment using logical connections via wired and/or wireless communications or data links to one or more remote computers. The remote computer(s) may be a workstation, a server computer, a router, a personal computer, a portable computer, a personal digital assistant, a cellular device, a microprocessor-based entertainment appliance, a peer device or other common network node, and may include many or all of the elements described relative to the computer. The logical connections or data links could include wired/wireless connectivity to a local area network (LAN) and/or larger networks, for example, a wide area network (WAN). Such LAN and WAN networking environments are commonplace in offices, and companies, and facilitate enterprise-wide computer networks, such as intranets, all of which may connect to a global communications network such as the Internet. For the purposes of the current disclosure a data link between two components may be any wired or wireless mechanism, medium, system and/or protocol between the two components, whether direct or indirect, that allows the two components to send and/or received data with each other.

The computer may be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi (such as IEEE 802.11x (a, b, g, n, etc.)) and Bluetooth™ wireless technologies. Thus, the communication may be a predefined structure as with a conventional network or simply an ad hoc communication between at least two devices.

The system may also include one or more server(s). The server(s) may also be hardware and/or software (e.g., threads, processes, computing devices). The servers may house threads to perform transformations by employing aspects of the invention, for example. The servers may be based locally or at a remote third party to create a cloud-based system. One possible communication between a client and a server may be in the form of a data packet adapted to be transmitted between two or more computer processes. The data packet may include a cookie and/or associated contextual information, for example. The system may include a communication framework (e.g., a global communication network such as the Internet) that may be employed to facilitate communications between the client(s) and the server(s).

While example embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages may exist even though they may not have been explicitly discussed herein.

What is claimed is:

1. A system for transforming patient medical record data into a visual and graphical indication of patient safety risk, comprising:
a communications device having a display and a graphical user interface;
a processor;
a patient database including one or more electronic health records comprising patient data of one or more patients; and
a memory coupled with the processor, the memory including instructions that, when executed by the processor, cause the processor to:
retrieve the one or more electronic health records of the one or more patients from the patient database;
classify the one or more patients into one or more risk categories, each comprising one or more data indicators; and
display a risk report on the communications device at one of an institutional level, a unit level, and a patient level;
wherein the risk categories include a situational awareness category, a patient-family experience category, a flow category, and a safety category;
wherein the processor remains in real-time communication with the patient database such that any updates to the one or more electronic health records are immediately reflected in the risk report;
wherein the institutional-level risk report further comprises:
a menu of toggleable risk category icons, wherein toggling one of the risk category icons filters the units by the toggled risk category;
a menu of toggleable data indicator icons, wherein the menu of toggleable data indicator icons appears when one of the risk category icons is toggled, and wherein toggling one of the data indicator icons further filters the units by the toggled data indicator; and
a plurality of unit representations, each unit representation corresponding to a unit and including a risk score for each risk category;
wherein the unit-level risk report further comprises:
a menu of toggleable risk category icons, wherein toggling one of the risk category icons filters the patients by the toggled risk category;
a menu of toggleable data indicator icons, wherein the menu of toggleable data indicator icons appears when one of the risk category icons is toggled, and wherein toggling one of the data indicator icons further filters the patients by data indicator;
a plurality of patient representations, each patient representation corresponding to a patient and including:
a risk score for each risk category; and
a data indicator display; and
wherein the patient-level risk report further comprises:
a risk score for each risk category;
a data indicator display;
one or more of the electronic health record of the patient and parts thereof; and
a selectable list of other patients on the unit.

2. The system of claim 1, wherein the risk categories further include a unit health category.

3. The system of claim 1, wherein the one or more data indicators of the situational awareness category include a plurality of the following:
a family concern for safety indicator;
a missed medication indicator;
a missed feeding indicator;
a cancelled surgery indicator;
a delayed procedure indicator;
a delayed diagnosis indicator;
an intravenous issue indicator;
a readmission indicator;
a high-risk therapies indicator;
a high pediatric early warning score indicator; and
a subjective degrading patient indicator.

4. The system of claim 1, wherein the one or more data indicators of the patient-family experience category include a plurality of the following:
a family concern for care indicator;
a surgical add-on indicator;
a communication indicator;
a multiple consults indicator;
a normally off-unit indicator;
a high pain score indicator;
a not-by-mouth indicator;
a length of stay indicator;
a transferred from different unit indicator;
a transferred from critical unit indicator; and
a previous experience failure indicator.

5. The system of claim 1, wherein the one or more data indicators of the flow category include a plurality of the following:
a pending admission indicator;
a predicted discharge indicator;
a medically ready discharges data indicator;
an overnight hold indicator; and
a unit capacity indicator.

6. The system of claim 1, wherein the one or more data indicators of the safety category include a plurality of the following:
an aggressive patient indicator;
a self-harm indicator;
a potential security risk indicator;
a line-drain-airway indicator;
a signed and held order indicator;
a prevention standards indicator;
a multiple medications indicator;
a reactive medication indicator;
a critical lines and tubes indicator;
a restraint order data indicator;
a seclusion order data indicator;
a private room order data indicator;
an imminent danger order data indicator;
a continuous observation order data indicator;
a multiple services indicator; and
a degrading patient indicator.

7. The system of claim 2, wherein the one or more data indicators of the unit health category include a plurality of the following:
a staffing level indicator;
a unit capacity indicator;
a staff on call indicator;
an isolation indicator; and
an overall unit risk score indicator.

8. The system of claim 1, wherein the risk report further comprises a bookmark icon associated with each patient;
wherein the bookmark icon in the patient-level risk report is located at one or more of on the selectable list of other patients on the unit and proximate to the risk scores;
wherein the bookmark icon in the unit-level risk report is located proximate to the risk scores;
wherein the bookmark icon is toggleable between an active state and an inactive state; and
wherein the institutional-level risk report and the unit-level risk report are filterable by bookmark icons toggled in the active state.

9. The system of claim 1, wherein the institutional-level risk report further comprises:
a header including information corresponding to institutional capacity; and
for each unit representation, unit information corresponding to total patients, total beds available, and patients temporarily off unit.

10. The system of claim 6, wherein the unit-level risk report further comprises:
a header including information corresponding to unit capacity;
a sub-header including information corresponding to patients temporarily off unit, expected admissions to the unit, and expected discharges from the unit;
a report button; and
in the patient representations, a prevention standards compliance icon for each patient classified under the prevention standards data indicator of the safety risk category;
wherein the prevention standards compliance icon indicates whether the patient is compliant with one or more prevention standards; and
wherein selecting the report button causes summarized information about the unit to be displayed in report form.

11. The system of claim 6, wherein the patient-level risk report further comprises:
a header; and
a prevention standards compliance icon for each patient classified under the prevention standards data indicator of the safety risk category;
wherein the prevention standards compliance icon indicates whether the patient is compliant with one or more prevention standards;
wherein the prevention standards compliance icon is selectable; and
wherein selecting the prevention standards compliance icon opens a window, said window providing details on the one or more prevention standards for the patient and compliance thereto.

12. The system of claim 1, wherein the patient-level risk report further comprises a means for receiving new patient data, and wherein the receipt of the new patient data causes the electronic health record of the patient to be updated with the received new patient data.

13. The system of claim 12, wherein updating the electronic health record with the received new patient data causes the processor to re-perform the retrieve, classify, and display steps in real time.

14. The system of claim 1, wherein the patient database further comprises medical center data, and wherein the processor further retrieves said medical center data.

15. The system of claim 1, wherein the institutional-level risk report and the unit-level risk report further comprise a search button.

16. A system for transforming patient medical record data into a visual and graphical indication of patient safety risk, comprising:
a communications device having a display and a graphical user interface;
a processor;
a patient database including medical center data and one or more electronic health records comprising patient data of one or more patients; and
a memory coupled with the processor, the memory including instructions that, when executed by the processor, cause the processor to:
retrieve the medical center data and the one or more electronic health records of the one or more patients from the patient database;
classify the one or more patients into one or more risk categories, each comprising one or more data indicators; and
display a risk report on the communications device at one of an institutional level, a unit level, and a patient level;
wherein the risk categories include a situational awareness category, a patient-family experience category, a flow category, and a safety category;
wherein the processor remains in real-time communication with the patient database such that any updates to the one or more electronic health records are immediately reflected in the risk report;
wherein the institutional-level risk report further comprises:
a menu of toggleable risk category icons, wherein toggling one of the risk category icons filters the units by the toggled risk category;
a menu of toggleable data indicator icons, wherein the menu of toggleable data indicator icons appears when one of the risk category icons is toggled, and wherein toggling one of the data indicator icons further filters the units by the toggled data indicator;
a header including information corresponding to institutional capacity; and
a plurality of unit representations, each unit representation corresponding to a unit and including:
a risk score for each risk category; and
unit information corresponding to total patients, total beds available, and patients temporarily off unit;
wherein the unit-level risk report further comprises:
a menu of toggleable risk category icons, wherein toggling one of the risk category icons filters the patients by the toggled risk category;
a menu of toggleable data indicator icons, wherein the menu of toggleable data indicator icons appears when one of the risk category icons is toggled, and wherein toggling one of the data indicator icons further filters the patients by data indicator;
a plurality of patient representations, each patient representation corresponding to a patient and including:
a risk score for each risk category; and
a data indicator display;
a header including information corresponding to unit capacity;
a report button wherein selecting the report button causes summarized information about the unit to be displayed in report form; and
a sub-header including information corresponding to patients temporarily off unit, expected admissions to the unit, and expected discharges from the unit; and
wherein the patient-level risk report further comprises:
a risk score for each risk category;
indicators corresponding to any data indicators under which each patient is classified;
one or more of the electronic health record of the patient and parts thereof; and
a selectable list of other patients on the unit.

17. The system of claim 16, wherein the one or more data indicators of the situational awareness category include a plurality of the following:
a family concern for safety indicator;
a missed medication indicator;
a missed feeding indicator;
a cancelled surgery indicator;
a delayed procedure indicator;
a delayed diagnosis indicator;
an intravenous issue indicator;
a readmission indicator;
a high-risk therapies indicator;
a high pediatric early warning score indicator; and
a subjective degrading patient indicator.

18. The system of claim 16, wherein the one or more data indicators of the patient-family experience category include a plurality of the following:
a family concern for care indicator;
a surgical add-on indicator;
a communication indicator;
a multiple consults indicator;
a normally off-unit indicator;
a high pain score indicator;
a not-by-mouth indicator;
a length of stay indicator;
a transferred from different unit indicator;
a transferred from critical unit indicator; and
a previous experience failure indicator.

19. The system of claim 16, wherein the one or more data indicators of the flow category include a plurality of the following:
a pending admission indicator;
a predicted discharge indicator;
a medically ready discharges data indicator;
an overnight hold indicator; and
a unit capacity indicator.

20. The system of claim 16, wherein the one or more data indicators of the safety category include a plurality of the following:
an aggressive patient indicator;
a self-harm indicator;
a potential security risk indicator;
a line-drain-airway indicator;
a signed and held order indicator;
a prevention standards indicator;
a multiple medications indicator;
a reactive medication indicator;
a critical lines and tubes indicator;
a restraint order data indicator;
a seclusion order data indicator;
a private room order data indicator;
an imminent danger order data indicator;
a continuous observation order data indicator;
a multiple services indicator; and
a degrading patient indicator.

21. The system of claim 16, wherein the one or more data indicators of the unit health category include a plurality of the following:
a staffing level indicator;
a unit capacity indicator;
a staff on call indicator;
an isolation indicator; and
an overall unit risk score indicator.

22. The system of claim 16, wherein the risk report further comprises a bookmark icon associated with each patient;
wherein the bookmark icon in the patient-level risk report is located at one or more of on the selectable list of other patients on the unit and proximate to the risk scores;
wherein the bookmark icon in the unit-level risk report is located proximate to the risk scores;
wherein the bookmark icon is toggleable between an active state and an inactive state; and
wherein the institutional-level risk report and the unit-level risk report are filterable by bookmark icons toggled in the active state.

23. The system of claim 20, wherein the unit-level risk report further comprises a prevention standards compliance icon for each patient classified under the prevention standards data indicator of the safety risk category, wherein the prevention standards compliance icon indicates whether the patient is compliant with one or more prevention standards.

24. The system of claim 20, wherein the patient-level risk report further comprises a prevention standards compliance icon for each patient classified under the prevention standards data indicator of the safety risk category;
wherein the prevention standards compliance icon indicates whether the patient is compliant with one or more prevention standards;
wherein the prevention standards compliance icon is selectable; and
wherein selecting the prevention standards compliance icon opens a window, said window providing details on the one or more prevention standards for the patient and compliance thereto.

25. The system of claim 16, wherein the patient-level risk report further comprises a means for receiving new patient data, and wherein the receipt of the new patient data causes the electronic health record of the patient to be updated with the received new patient data.

26. The system of claim 25, wherein updating the electronic health record with the received new patient data causes the processor to re-perform the retrieve, classify, and display steps in real time.

27. The system of claim 16, wherein the institutional-level risk report and the unit-level risk report further comprise a search button.
